(19) United States
(12) Patent Application Publication
(10) Pub. No.: US 2026/0171240 A1
(43) Pub. Date: Jun. 18, 2026

(54) METHOD FOR PREDICTING THE RISK OF THE OCCURRENCE OF SUDDEN DEATH AND ASSOCIATED DEVICES

(71) Applicants: INSTITUT NATIONAL DE LA SANTE ET DE LA RECHERCHE MEDICALE, Paris Cedex 13 (FR); UNIVERSITE PARIS CITE, Paris (FR); ASSISTANCE PUBLIQUE HOPITAUX DE PARIS, Paris (FR); GROUPE DES ECOLES NATIONALES D'ECONOMIE ET STATISTIQUE, Palaiseau (FR); MEDYKAL AG, Zug (CH)

(72) Inventors: Xavier JOUVEN, Clamart (FR); Younes YOUSSFI, Paris (FR); Wulfran BOUGOUIN, Massy (FR)

(21) Appl. No.: 18/712,487

(22) PCT Filed: Nov. 23, 2022
(86) PCT No.: PCT/EP2022/082993

§ 371 Date: May 22, 2024

(30) Foreign Application Priority Data
Nov. 23, 2021 (FR) FR2112396

(51) Int. Cl. G16H 50/20; G16H 50/30; G16H 50/70

(52) U.S. Cl. CPC G16H 50/20; G16H 50/30; G16H 50/70

(57) ABSTRACT

The present invention relates to a method for predicting the risk of the occurrence of sudden death in a patient, the method being computer implemented and comprising the steps of:
receiving data relating to the care pathway of a patient,
determining from the received data whether the patient belongs to one of a set of predefined groups, each group being associated with a set of predefined data,
searching, for each predefined determined data, of the value of the predefined data for the patient, to obtain a set of values specific to the patient, and
applying a neural network to the patient-specific values to obtain a risk of the occurrence of sudden death for the patient, the neural network being specific to the determined group.

This patent application claims the benefit of document FR 21/12396 filed on Nov. 23, 2021 which is hereby incorporated by reference.

### TECHNICAL FIELD OF THE INVENTION

The present invention relates to a method for predicting the risk of the occurrence of a sudden death in a patient. The present invention also relates to a computer program product and a readable information carrier involved in the implementation of the prediction method.

### TECHNOLOGICAL BACKGROUND OF THE INVENTION

Sudden death is defined as an unexpected death without obvious extracardiac cause, occurring with rapid collapse in the presence of a bystander, or in the absence of a bystander occurring within one hour after the onset of symptoms. This pathology affects 30,000 to 40,000 people per year in France, and about 300,000 people per year in Europe.

The prognosis remains extremely poor, with a survival rate of less than 10% in several recent studies. Several tools have been proposed to improve prognosis, concerning prehospital management, notably via the chain of survival, early cardiac massage by bystanders, early defibrillation, or hospital management (via early coronary management or the application of therapeutic hypothermia).

Nevertheless, survival results remain disappointing, despite a recent improvement according to some studies.

Considering these modest results on the therapeutic side, several preventive alternatives have been proposed to prevent the occurrence of such events. Thus, the development of antiarrhythmic treatments and automatic implantable defibrillators has allowed significant prevention in patients identified as being at high risk of sudden death.

Optimization of the use of these preventive treatments therefore depends on the identification of patients at risk.

In this context, although a lot of research has been conducted on the “post-event” side, particularly in the care of patients who have suffered a sudden death, predicting the occurrence of such an event remains difficult. The identification of at-risk patients therefore remains a major research challenge, with disappointing results to date.

Certain groups of patients at very high risk of sudden death have been identified (specific structural or electrical pro-arrhythmogenic heart disease) and are already receiving specialized rhythmology management.

However, from an epidemiological point of view, these constitute only a very small fraction of the total population concerned, and the vast majority of patients who suffer sudden death are not part of these populations. Indeed, the main cause of sudden death remains ischemic heart disease, either during an acute event (myocardial infarction) or during the follow-up of these patients. The cohort of patients with ischemic heart disease is very large, and only a small proportion of them will experience sudden death during the course of the disease.

Therefore, there is a discrepancy between a very high-risk but small population (specific pro-arrhythmic, structural, or electrical heart disease) and a low risk but very large population (ischemic heart disease), which therefore constitutes the bulk of sudden death patients in the general population.

The challenge of predicting sudden death therefore remains, since most patients cannot benefit from individual risk stratification, in the absence of clearly identified population risk factors (unlike global cardiovascular risk, for example, for which tools such as the Framingham score allow individual risk assessment).

Some risk factors, including family history, have been proposed to stratify this individual risk. In the population of patients with ischemic heart disease, which constitutes the majority of sudden death victims, prediction is currently based essentially on the left ventricular ejection fraction, with relatively disappointing results.

### SUMMARY OF THE INVENTION

There is therefore a need for a method to predict the risk of the occurrence of sudden death in a patient.

To this end, the description describes a method for predicting the risk of occurrence of sudden death in a patient, the method being computer-implemented and comprising the steps of receiving data related to the care pathway of a patient, determining from the data related to the care pathway whether the patient belongs to one of a set of predefined groups, each group being associated with a set of predefined data. In order to obtain a determined group and a set of determined predefined data, searching, for each determined predefined data, for the value of the predefined data for the patient, in order to obtain a set of values specific to the patient, and applying a neural network to the values specific to the patient in order to obtain a risk of the occurrence of sudden death in the patient, the neural network being specific to the determined group.

The present method differs from a machine learning method for the analysis of the electrocardiogram signal or populations at high cardiovascular risk. Indeed, this type of methods is limited to populations at cardiovascular risk.

By contrast, the present invention proposes a prediction on the general population and not only on populations at cardiovascular risk. Indeed, the populations at cardiovascular risk represent only 5% of the population whereas it is necessary to be able to predict the risk of sudden death on the other 95%.

This leads to the identification of new groups, the 8 groups mentioned in the present application. To date, none of these groups had been so identified.

For example, the present invention makes it possible to tell whether you are more at risk of dying of sudden death if you have not had your annual dental scaling or are taking psychotropic drugs.

The method thus makes it possible to identify people with a high probability of rapidly dying suddenly and requiring implantation of an implanted defibrillator. These people are for the most part not the people classically considered for sudden death since they are not part of the populations at cardiovascular risk. Yet they represent 90% of the total population at risk of sudden death.

The present process therefore makes it possible to fight effectively against the scourge of sudden death.

According to particular embodiments, the prediction method presents one or more of the following features, taken alone or in any technically possible combination:
each neural network is a gated recurrent neural network.
the received data are data related to the care pathway of the patient during the previous five years.
each predefined data is the presence of a disorder or the intake of a drug.
the number of predefined data of a group is between 10 and 30, preferably between 15 and 25, advantageously equal to 20.
a group is associated with predefined data related to a breathing disorder or to the intake of a product limiting breathing disorders.
a group is associated with predefined data related to a neurological disorder or to the intake of a product limiting neurological disorders.
a group is associated with predefined data related to a cancer disorder or to the intake of a product limiting cancer disorders.
a group is associated with predefined data related to an addiction disorder or to the intake of a product limiting addiction disorders.
a group is associated with predefined data related to an aging disorder or to the intake of a product limiting aging disorders.
a group is associated with predefined data related to a cardiac disorder or to the intake of a product limiting cardiac disorders.
the groups are obtained by applying a k-means partitioning technique on a set of data comprising data related to the care pathway of a set of patients and each predefined data of a group is obtained by applying a language analysis tool on a set of data comprising data related to the care pathway of the set of patients.
the set of data includes artificial data generated by applying a function on data related to the care pathway of a set of patients.
the function is an adversarial neural network.

The description also describes a computer program product including program instructions forming a computer program stored on a readable information medium, the computer program being loadable onto a data processing unit and implementing an evaluation method such as previously described when the computer program is implemented on the data processing unit.

The description also relates to a readable medium of information including program instructions forming a computer program, the computer program being loadable onto a data processing unit and implementing an evaluation method as previously described when the computer program is implemented on the data processing unit.

### BRIEF DESCRIPTION OF THE FIGURES

Features and advantages of the invention will become apparent from the following description, which is given only as a non-limiting example, and is made with reference to the attached drawings, in which:

FIG. 1 is a schematic representation of a system and a computer program product, and

FIG. 2 is a flowchart of an example implementation of a method for predicting the risk of the occurrence of a sudden death in a patient.

### DETAILED DESCRIPTION OF PREFERRED EMBODIMENTS

### Description of the System Used

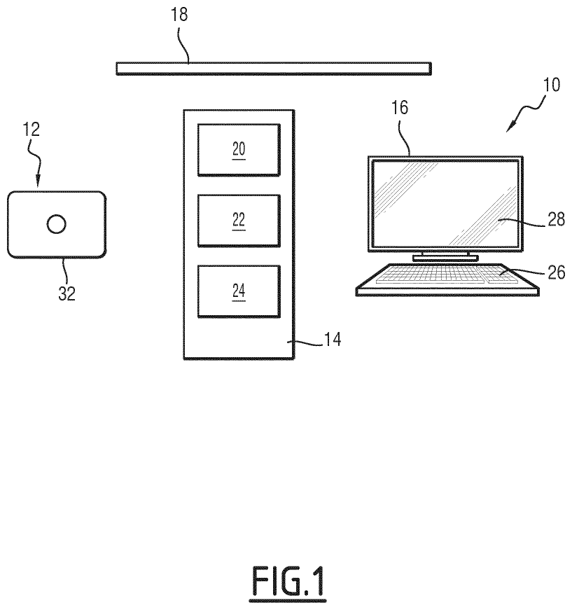

A system 10 and a computer program product 12 are shown in FIG. 1.

The interaction between the system 10 and the computer program product 12 enables the implementation of a method for predicting the risk of the occurrence of sudden death in a patient. The prediction method is thus a computer-implemented method.

The system 10 is a desktop computer. Alternatively, the system 10 is a rack-mounted computer, a laptop computer, a tablet, a personal digital assistant (PDA) or a smartphone.

In the case of FIG. 1, the system 10 comprises a computer 14, a user interface 16, and a communication device 18.

The computer 14 is an electronic circuit designed to manipulate and/or transform data represented by electronic or physical quantities in registers of the system 10 and/or memories into other similar data corresponding to physical data in the memories of registers or other types of display devices, transmission devices, or storage devices.

As specific examples, the computer 14 comprises a single-core or multi-core processor (such as a central processing unit (CPU), a graphics processing unit (GPU), a microcontroller, and a digital signal processor (DSP)), a programmable logic circuit (such as an application-specific integrated circuit (ASIC), a field programmable gate array (FPGA), a programmable logic device (PLD), and programmable logic arrays (PLAs)), a state machine, a logic gate, and discrete hardware components.

The computer 14 comprises a data processing unit 20 able to process data, in particular by performing calculations, memories 22 able to store data, and a reader 24 able to read a computer readable medium.

The user interface 16 comprises an input device 26 and an output device 28.

The input device 26 is a device for the user of the system 10 to enter information or commands into the system 10.

In FIG. 1, the input device 26 is a keyboard. Alternatively, the input device 26 is a pointing device (such as a mouse, touchpad, and graphics tablet), a voice recognition device, an eye tracker, or a haptic (motion analysis) device.

The output device 28 is a graphical user interface, that is, a display unit designed to provide information to the user of the system 10.

In FIG. 1, the output device 28 is a display screen for visual presentation of the output. In other embodiments, the output device 28 is a printer, an augmented and/or virtual display unit, a speaker or other sound generating device for presenting the output in sound form, a vibration and/or odor producing unit, or a unit able to produce an electrical signal.

In one specific embodiment, the input device 26 and the output device 28 are the same component forming a Human Machine Interface, such as an interactive screen.

The communication device 18 allows for one way or two way communication between the components of the system 10. For example, the communication device 18 is a bus communication system or an input/output interface.

The presence of the communication device 18 allows, in some embodiments, the components of the computer 14 to be remote from each other.

The computer program product 12 comprises a computer readable medium 30.

The computer-readable medium 30 is a tangible device readable by the reader 14 of the computer 14.

Notably, the computer readable medium 30 is not a transient signal per se, such as radio waves or other freely propagating electromagnetic waves, such as light pulses or electronic signals.

Such a computer-readable storage medium 30 is, for example, an electronic storage device, a magnetic storage device, an optical storage device, an electromagnetic storage device, a semiconductor storage device or any combination thereof.

As a non-exhaustive list of more specific examples, the computer-readable storage medium 30 is a mechanically encoded device, such as punched cards or embossed structures in a groove, a floppy disk, a hard disk, a read-only memory (ROM), a random access memory (RAM), an erasable programmable read-only memory (EPROM), an electrically erasable and readable memory (EEPROM), a magneto-optical disk, a static random access memory (SRAM), a compact disk (CD-ROM), a digital versatile disk (DVD), a USB flash drive, a floppy disk, a flash memory, a solid-state drive (SSD), or a PC card such as a PCMCIA memory card.

A computer program is stored on the computer readable storage medium 30. The computer program includes one or more sequences of stored program instructions.

Such program instructions, when executed by the data processing unit 20, cause steps of the estimation method to be performed.

For example, the form of the program instructions is a source code form, a computer executable form, or any intermediate form between a source code and a computer executable form, such as the form resulting from conversion of the source code via an interpreter, an assembler, a compiler, a linker, or a localizer. Alternatively, the program instructions are a microcode, firmware instructions, state definition data, integrated circuit configuration data (for example, VHDL) or object code.

The program instructions are written in any combination of one or more languages, for example, an object-oriented programming language (FORTRAN, C++, JAVA, HTML), a procedural programming language (for example, C language).

Alternatively, the program instructions are downloaded from an external source via a network, as is the case for applications. In this case, the computer program product comprises a computer-readable data carrier on which the program instructions are stored or a data carrier signal on which the program instructions are encoded.

In each case, the computer program product 12 comprises instructions that can be loaded into the data processing unit 20 and able to cause the prediction method to be executed when executed by the data processing unit 20. According to the embodiments, the execution is performed entirely or partially either on the system 10, namely, a single computer, or in a distributed system among multiple computers (in particular via the use of cloud computing).

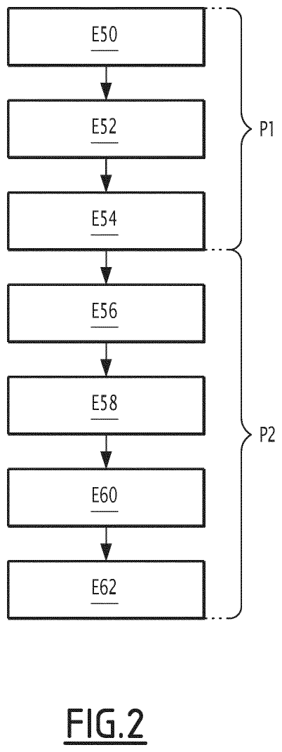

The operation of the system 10 is now described with reference to FIG. 2, which is a flowchart illustrating an example implementation of the prediction method.

The method is a method for predicting the risk of occurrence of sudden death in a patient.

As a non-limiting example, the patient is an adult human patient.

The human subject has any medical profile. The subject may, in particular, not be at cardiovascular risk.

The method is intended to apply to any type of population.

The method comprises two phases, a preparation phase P1 and an operation phase P2.

According to the case, the preparation phase P1 is implemented by the system 10 or by another system. Generally, the preparation phase P1 is implemented well before the exploitation phase P2.

In this case, as will be described later, the preparation phase P1 includes three steps: a training step E50, a determination step E52 and a training step E54.

The operation phase P2, will now be described, which includes a reception step E56, a determination step E58, a search step E60 and an application step E62.

During the reception step E56, the system 10 receives data relating to the care history of the patient over the previous five years.

The data of the patient is therefore data of care.

The data relating to the care pathway lists the hospital stays of the patient, the disorders detected in the patient, the examinations carried out and the treatments applied.

In France, this data is available through a public organization, typically social security or the Regional Health Agency.

Nevertheless, it could be envisaged that data relating to the care pathway be obtained using the answers from the patient to questions relating to their previous care pathway.

The period of 5 years is chosen because the Applicant has shown that a shorter period leads to less reliable predictions and that a longer period does not increase the reliability of predictions.

It can be specified here that the data relating to the health course is not recorded signal data, such as an electrocardiogram signal. Only the results of interpretation are taken into account.

Additionally, health journey data is broader than a collection of recorded heart disease signals.

In particular, the data relating to the health course includes data concerning any type of pathology. For example, health journey data will indicate when scalings have occurred as well as lab results.

In addition, data relating to health pathways are heterogeneous in the sense that they bring together data of different natures.

The data relating to the health course thus includes data chosen from the following classes: medication taken, history, doctor's visit, list of pathologies, laboratory results, intervention of the firefighters, visit to the emergency room and so on.

Preferably, the data relating to the health course includes all the previous classes.

During the determination step E58, the system 10 searches among a set of predefined groups the group to which the patient belongs.

To do this, the system 10 applies a classification function to the data received at reception step E50 to determine the group that is closest.

The groups are predefined groups that present relatively similar behaviors relative to the risk of the occurrence of sudden death.

The groups were obtained by applying a k-means partitioning technique on a set of data comprising care pathway data of a set of patients.

The set of data is, in the experiments of the Applicant, representative of the general population.

The set of data also includes sufficient data related to the occurrence of sudden death to allow the groups to be obtained.

In the presence of an insufficient or unbalanced sample (under-representation of sudden death cases), the set of data includes artificial data generated by applying a function on data related to the care pathway of a set of patients.

According to a particular example, the function is an adversarial neural network that is able to generate from care pathway data.

Furthermore, each group is associated with a predefined set of data.

In the example described, each predefined data is the presence of a disorder or the intake (administration) of a product.

Each predefined data of a group is obtained by applying a language analysis tool on a set of data comprising data related to the care pathway of the set of patients.

The number of predefined data of a group is between 10 and 30, preferably between 15 and 25, advantageously equal to 20.

Furthermore, preferably, the amount of data of the type “presence of a disorder” and the amount of data of the type “product administered” are equal.

According to the described example, the predefined groups comprise:
a group associated with predefined data related to a breathing disorder or the intake of a drug limiting breathing disorders,
a group associated with predefined data related to a neurological disorder or the use of a drug limiting neurological disorders,
a group associated with predefined data related to a cancer disorder or the use of a drug limiting cancer disorders,
a group associated with predefined data related to an addiction disorder or the use of a drug limiting addiction disorders, and
a group associated with predefined data related to an aging disorder or to the use of a drug limiting aging disorders, and
a group is associated with predefined data related to a cardiac disorder or use of a drug limiting cardiac disorders.

In some cases, there may be multiple groups addressing the same disorder. In particular, it may be favorable, as shown by the experience of the applicant, to have two groups addressing cardiac disorders.

In each case, the groups and the predefined data are obtained by implementing the reception step E50 of forming a database and the step of determining the groups and predefined data E52.

During a search step E60, the system 10 searches for each determined predefined data, the value of the predefined data for the patient, to obtain a set of values specific to the patient.

Such a search can, for example, be reduced to an extraction of the data of the care pathway.

The term “value” is here understood in a broad sense as including both binary values (took the drug or not) or quantification values (typically a scale between 1 and 10 for pain).

Alternatively, or in addition, these values can be obtained through questions to the patient or to care personnel.

In application step E62, the system 10 applies a neural network to the patient-specific values to obtain a risk of the occurrence of sudden death in the patient, the neural network being specific to the determined group.

The neural network has previously learned, in particular, by using the same data that allowed the predefined groups to be found. This corresponds to the training step E54 of the first phase P1.

This means that the system 10 has in memory the attributes of each predefined group as well as the specific neural networks.

These neural networks are specific in that their inputs are the values of the predefined data associated with the group considered.

Typically, for the group associated with predefined data related to a breathing disorder or to the intake of a drug limiting the breathing disorders, the neural network will take as input, 10 values of predefined data related to a breathing disorder and 10 values of intake of a drug limiting the breathing disorders.

Furthermore, in the example described, each neural network is a recurrent gated neural network.

The probability value is for example expressed as a score for the next three months.

However, any form of score can be considered to give the result of the neural network calculation.

The method can thus effectively predict the risk of the occurrence of sudden death.

This is shown in the next section.

### EXPERIMENTAL RESULTS

The present method has been the subject of experiments by the Applicant, which are now described.

### Objective of the Experiments

The main objective of these experiments is to predict the occurrence of sudden adult death, by comparing data from cases (patients, victim of sudden death) and from four control populations:
population 1: patients with ischemic heart disease without sudden death,
population 2: patients with acute coronary syndrome without sudden death
population 3: patients with chronic heart failure without sudden death, and
population 4: individuals representative of the general population.

These experiments used the registry of the Centre d'Expertise de la Mort Subite (Sudden Death Expertise Center) and the medico-administrative databases of the Assurance Maladie (Health Care System). Their scientific interest has been evaluated by the French Society of Cardiology, which has emphasized their public health interest and the major contribution they constitute.

These experiments sought to build groups of patients within the sudden death population, to identify and represent the heterogeneity of this population and the risk factors associated with them, and to develop a prediction algorithm for sudden death, based on the care trajectories observed before the event.

### Data Used

### Description of the Study Cohort

### Cases Included

Since May 2011, the Sudden Death Expertise Center has been collecting all cases of sudden death occurring in a given geographic area (Paris and the 3 adjacent departments, Hauts de Seine, Seine-Saint-Denis, and Val de Mane), which represents a total population of 6.6 million inhabitants, namely, 10% of the French population. This collection was made possible by a tiered collaboration between the prehospital emergency services (Paris Fire Brigade, SAMU), hospitals (resuscitation and cardiology departments) and the Paris Forensic Institute.

For all the cases included, information relating to the occurrence of the event (Utstein criteria), on the management (pre and intra hospital) and on the outcome of the patients (in terms of survival and neurological prognosis) were collected prospectively, with multiple sources and frequent quality controls (allowing to evaluate the exhaustiveness to 99% of the cases in the area of interest). This collection received a favorable opinion from the Comité consultatif sur le traitement de l'information en matiėre de recherche (CCTIRS, (Consultative Committee for the Treatment of Information in Research studies) file N12.336) and an authorization from the CNIL (National Committee for Information and Liberty) (decision DR-2012-445).

The case population of our study therefore consists of patients included in the CEMS registry who presented a sudden death between May 15, 2011, and Dec. 31, 2020. Over the considered study period of 9 years (2011-2020), this represents 24,000 included cases.

### Control Populations

The control populations were defined and collected from the medico-administrative databases of the Assurance Maladie. These populations contain 4 different cohorts of controls, with for each cohort a matching of 3 controls for 1 case, matched on sex, age and department of residence. 288,000 control individuals were thus included in these experiments.

For the first population (patients with ischemic heart disease), in order to perform a 3:1 matching of controls to cases, 72,000 control individuals were selected from this population. The identification of patients with ischemic heart disease was performed according to a previously described medical mapping method.

The same methodology was followed for the second population (patients with acute coronary syndrome) and third population (patients with chronic heart failure).

For the fourth population (individuals, representative of the general population), a control group, representative of the general population, was constituted with 3 controls for 1 case. A total of 72,000 controls were randomly selected in Paris and the 3 adjacent departments (Hauts de Seine, Seine-Saint-Denis, and Val de Marne), excluding individuals previously included in the 3 previously defined populations.

### Description of the Data Used

Within the framework of these experiments, the medical history of the 24,000 sudden death cases and 288,000 control individuals described above, was collected over a period of 5 to 10 years before the event. This information was extracted from the medico-administrative databases of the Assurance Maladie, from the Système National des Données de Santé (SNDS).

The SNDS is a warehouse of pseudo-anonymized medico-administrative data covering the entire French population and containing all care presented for reimbursement. It is managed by the Caisse Nationale de l'Assurance Maladie (CNAM) and links data from the Assurance Maladie (SNIIRAM database), hospital data (PMSI database) and medical causes of death (INSERM CépiDC database).

It currently contains more than 3,000 variables and represents an annual flow of 1.2 billion health care forms, 11 million hospital stays and 500 million medical procedures.

The data analyzed in this study correspond to all individual care and medical consumption data that have given rise to reimbursement: medical examinations and devices, hospitalizations, drugs and long-term illnesses.

### Statistical Methods

### Classification of the Sudden Death Population

The Applicant has developed a classification model (more often referred to as “clustering”) of the sudden death population, by exploiting the care trajectories observed over a period of 5 years prior to the cardiac arrest.

For this purpose, an unsupervised clustering algorithm is used. This makes it possible to identify and represent the heterogeneity of sudden death cases and the risk factors associated with them.

More specifically, this algorithm involves the use of a language analysis tool followed by the use of a k-means partitioning algorithm.

The language analysis tool is used to represent patients based on the time period information contained in their care trajectory. In this case, the Applicant used the Word2Vec algorithm.

The k-means partitioning algorithm is more often referred to as “k-means”. The algorithm identifies groups (“clusters”) that are relevant to the prediction of sudden death.

In the present case, this led the Applicant to identify seven clusters whose characteristics are explained below.

### Group 1:

The first group is characterized by the following elements:

TABLE 1

| Size | Group 1 | Total population |
|---|---|---|
| Number of cases | 3,791 | 30.0% |
| Age | 78 | 68 |
| Male | 45% | 61% |
| Universal health insurance | 1% | 4.1% |
| (CMU) | | |
| Shockable rhythm | 10.9% | 16.8% |
| Transported alive | 17.4% | 24% |
| Mortality rate (after | 96.1% | 94.6% |
| hospitalization) | | |

TABLE 2

| | Deviation from |
|---|---|
| | average |
| Diagnosis | |
| Hypertension | +14.4 |
| Fibrillation | +7.5 |
| Senile cataract | +7.4 |
| Motor abnormalities (difficulty walking) | +6.4 |
| Chronic heart failure | +5.7 |
| Dependency (in assistance and care) | +5.6 |
| Alzheimer's disease | +4.7 |
| Hyperthyroidism | +4.4 |
| Chronic renal failure | +4.0 |
| Dementia | +3.8 |
| Products administered | |
| Viral Vaccines | +18.3 |
| Vitamins A and D | +16.5 |
| Laxatives | +16.4 |
| Calcium | +14.7 |
| Anti-infectives | +13.7 |
| Drugs affecting bone structure and mineralization | +13.3 |
| Anti-thrombotic | +13.3 |
| Ophthalmic products (excluding vitamin A) | +12.4 |
| Combination of anti-inflammatory and anti-infective | +12.3 |
| Loop diuretics | +11.8 |

TABLE 3

| Initial heart rate | % |
|---|---|
| Ventricular fibrillation (VF) | 10.7 |
| Ventricular tachycardia (VT) | 0.2 |
| Other | 77.5 |

TABLE 4

| Causes of death (for 657 cases) | % |
|---|---|
| Not known | 5.4 |
| Hypoxia | 4.4 |
| Ischemia | 3.5 |
| Heart disease (non-ischemic) | 1.7 |
| Other | 0.6 |
| Pulmonary embolism | 0.6 |
| Subarachnoid hemorrhage | 0.4 |
| Trauma | 0.4 |
| Dyskalemia | 0.3 |
| Drug poisoning | 0.0 |

### Group 2:

The second group is characterized by the following elements:

TABLE 5

| Size | Group 2 | Total population |
|---|---|---|
| Number of cases | 3,501 | 27.7% |
| Age | 74 | 68 |
| Male | 75% | 61% |
| Universal health insurance | 1.6% | 4.1% |
| (CMU) | | |
| Shockable rhythm | 18.7% | 16.8% |
| Transported alive | 24.5% | 24% |
| Mortality rate (after | 94.6% | 94.6% |
| hospitalization) | | |

TABLE 6

| | Deviation from |
|---|---|
| Diagnosis | average |
| Diabetes mellitus without insulin dependence | +19.9 |
| Hypertension | +18.9 |
| Ischemic heart disease | +17.7 |
| Lipoprotein metabolism disorders and other | +15.1 |
| dyslipidemias | |
| Presence of implants and grafts in the heart | +12.2 |
| Chronic heart failure | +11 |
| Fibrillation | +9.5 |
| Diabetes mellitus with insulin dependence | +9.5 |
| Angina | +7.8 |
| Atherosclerosis | +7.5 |
| | Deviation from |
| Products Administered | Average |
| Non-associated serum lipid lowering agents | +31.6 |
| Beta-blocking agents | +27.3 |
| Blood glucose lowering drugs other than insulin | +26.2 |
| Anti-thrombotic | +23.9 |
| Angiotensin-converting enzyme (ACE) inhibitors | +23 |
| Selective calcium channel blockers with vascular | +17.4 |
| effects | |
| Loop diuretics | +15.9 |
| Angiotensin II antagonists | +14.3 |
| Vasodilators used in heart disease | +14.3 |
| Combinations of angiotensin II antagonists | +14.1 |

TABLE 7

| Initial heart rate | % |
|---|---|
| Ventricular fibrillation (VF) | 18.3 |
| Ventricular tachycardia (VT) | 0.4 |
| Other | 71.9 |

TABLE 8

| Causes of death (for 856 cases) | % |
|---|---|
| Not known | 7.2 |
| Ischemia | 6.5 |
| Heart disease (non-ischemic) | 4.3 |
| Hypoxia | 3.8 |
| Other | 0.7 |
| Subarachnoid hemorrhage | 0.6 |
| Dyskalemia | 0.5 |
| Pulmonary embolism | 0.5 |
| Trauma | 0.3 |
| Drug poisoning | 0.0 |

### Group 3:

The third group is characterized by the following elements:

TABLE 9

| Size | Group 3 | Total population |
|---|---|---|
| Number of cases | 2,285 | 18.1% |
| Age | 52 | 68 |
| Male | 68% | 61% |
| Universal health insurance | 7.2% | 4.1% |
| (CMU) | | |
| Shockable rhythm | 23.8% | 16.8% |
| Transported alive | 34.5% | 24% |
| Mortality rate (after | 90.4% | 94.6% |
| hospitalization) | | |

TABLE 10

| | Deviation from |
|---|---|
| Diagnosis | average |
| Hypertension | −29.3 |
| Chronic heart failure | −14.1 |
| Ischemic heart disease | −14.0 |
| Fibrillation | −14.0 |
| Lipoprotein metabolism disorders and other | −14.0 |
| dyslipidemias | |
| Diabetes mellitus without insulin dependence | −14.0 |
| Presence of implants and grafts in the heart | −10.2 |
| Senile cataract | −9.2 |
| Respiratory failure, not classified in other categories | −8.7 |
| Chronic renal failure | −8.6 |
| | Deviation from |
| Products Administered | Average |
| Anti-thrombotic | −34.3 |
| Serum lipid reducing agents | −32.1 |
| Viral vaccines | −29.8 |
| Beta-blocking agents | −27.4 |
| Loop diuretics | −26.1 |
| Selective calcium channel blockers with vascular | −22.9 |
| effects | |
| Angiotensin converting enzyme (ACE) inhibitors | −21.6 |
| Laxatives | −21.3 |
| Angiotensin II antagonists | −18.8 |
| Antidepressants | −17.9 |

TABLE 11

| Initial heart rate | % |
|---|---|
| Ventricular fibrillation (VF) | 23.0 |
| Ventricular tachycardia | 1.3 |
| Other | 68.5 |

TABLE 12

| Causes of death (for 854 cases) | % |
|---|---|
| Ischemia | 11.6 |
| Not known | 11.1 |
| Hypoxia | 3.9 |
| Heart disease (non-ischemic) | 3.5 |
| Trauma | 2.8 |
| Other | 1.4 |
| Subarachnoid hemorrhage | 1.4 |
| Pulmonary embolism | 1.0 |
| Dyskalemia | 0.6 |
| Drug poisoning | 0.2 |

### Group 4:

The fourth group is characterized by the following elements:

TABLE 13

| Size | Group 4 | Total population |
|---|---|---|
| Number of cases | 1,383 | 10.9% |
| Age | 59 | 68 |
| Male | 51% | 61% |
| Universal health insurance | 9.6% | 4.1% |
| coverage (CMU) | | |
| Shockable rhythm | 7.6% | 16.8% |
| Transported alive | 23.7% | 24% |
| Mortality rate (after | 96.2% | 94.6% |
| hospitalization) | | |

TABLE 14

| | Deviation from |
|---|---|
| | average |
| Diagnosis | |
| Depressive episodes | 12.6 |
| Mental disorders related to alcohol consumption | 11.8 |
| Intoxication with anti-epileptics, sedatives, hypnotics | 9.3 |
| and anti-Parkinson drugs | |
| Psychotropic drug intoxication, not elsewhere | 6.3 |
| classified | |
| Bipolar affective disorder | 5 |
| Epilepsy | 4.8 |
| Schizophrenia | 4.7 |
| Self-poisoning with anti-epileptics, sedatives, | 4.5 |
| hypnotics, anti-Parkinsonian drugs and psychotropic | |
| drugs | |
| Self-harm by unspecified means | 4.3 |
| Organic psychosis, unspecified | 4.2 |
| Products administered | |
| Antipsychotics | 49.8 |
| Antidepressants | 35.6 |
| Sedatives and hypnotics | 33.3 |
| Anxiolytics | 28.5 |
| Anticholinergic agents | 24.3 |
| Antiepileptics | 22.1 |
| Drugs used in addiction related disorders | 14 |
| Other mineral supplements (other than potassium | 9.1 |
| and calcium) | |
| Other products for the digestive tract and | 7.7 |
| metabolism | |
| Centrally acting muscle relaxants | 6.3 |

TABLE 15

| Initial heart rate | % |
|---|---|
| Ventricular fibrillation (VF) | 7.2 |
| Ventricular tachycardia (VT) | 0.4 |
| Other | 82.2 |

TABLE 16

| Causes of death (for 334 cases) | % |
|---|---|
| Hypoxia | 9.0 |
| Not known | 5.8 |
| Ischemia | 2.1 |
| Other | 1.7 |
| Heart disease (non-ischemic) | 1.3 |
| Pulmonary embolism | 1.1 |
| Drug poisoning | 1.1 |
| Trauma | 1.0 |
| Subarachnoid hemorrhage | 0.6 |
| Dyskalemia | 0.2 |

### Group 5:

The fifth group is characterized by the following elements:

TABLE 17

| Size | Group 5 | Total population |
|---|---|---|
| Number of cases | 640 | 5.1% |
| Age | 70 | 68 |
| Male | 63% | 61% |
| CMU | 3.5% | 4.1% |
| Shockable rhythm | 9.1% | 16.8% |
| Transported alive | 24.1% | 24% |
| Mortality rate (after | 94.7% | 94.6% |
| hospitalization) | | |

TABLE 18

| | Deviation from |
|---|---|
| | average |
| Diagnosis | |
| Lung disease with chronic obstruction | 38.2 |
| Respiratory failure | 33.7 |
| Asthma | 26 |
| Dependence on a machine or auxiliary equipment | 12.6 |
| Bacterial pneumonia | 11 |
| Acute bronchitis | 10.6 |
| Emphysema | 10.1 |
| Pneumonia with unspecified microorganism | 9.4 |
| Mental and behavioral disorders related to | 9.3 |
| tobacco use | |
| Respiratory abnormalities | 8.5 |
| Products administered | |
| Adrenergic inhalants | 65.6 |
| Other inhaled medical products for obstructive | 62.5 |
| airway diseases | |
| Other systemic drugs for airway obstructive | 34.8 |
| diseases | |
| Non-associated systemic corticosteroid | 30.9 |
| Bacterial vaccines | 23.8 |
| Macrolides, lincosamides and streptogramins | 22.4 |
| Viral vaccines | 19.4 |
| Other beta-lactam antibiotics | 16.8 |
| Antihistamines for systemic use | 14.3 |
| Expectorants, except in combination with cough | 13.6 |
| suppressants | |

TABLE 19

| Initial heart rate | % |
|---|---|
| Ventricular fibrillation (VF) | 8.8 |
| Ventricular tachycardia (VT) | 0.3 |
| Other | 79.6 |

TABLE 20

| Causes of death (for 158 cases) | % |
|---|---|
| Hypoxia | 12.2 |
| Not known | 5.5 |
| Ischemia | 3.9 |
| Heart disease (non-ischemic) | 1.2 |
| Other | 0.8 |
| Subarachnoid hemorrhage | 0.5 |
| Trauma | 0.5 |
| Dyskalemia | 0.2 |

### Group 6:

The sixth group is characterized by the following elements:

TABLE 21

| Size | Group 6 | Total population |
|---|---|---|
| Number of cases | 549 | 4.7% |
| Age | 60 | 68 |
| Male | 61% | 61% |
| Universal health insurance (CMU) | 4.2% | 4.1% |
| Shockable rhythm | 8.0% | 16.8% |
| Transported alive | 16.2% | 24% |
| Mortality rate (after hospitalization) | 98.5% | 94.6% |

TABLE 22

| | Deviation from |
|---|---|
| | average |
| Diagnosis | |
| Chemotherapy and radiotherapy | 78.4 |
| Adjustment and maintenance of an internal | 52.6 |
| prosthesis | |
| Secondary malignant tumor of digestive or | 48.2 |
| respiratory organs | |
| Malignant tumor of lymph nodes | 34.4 |
| Secondary malignant neoplasm of other sites | 34.0 |
| Malignant tumor of bronchus and lung | 23 |
| Anemia during tumor disease | 22 |
| Pain (not classified elsewhere) | 19.5 |
| Personal history of medical treatment | 16.1 |
| Malaise and fatigue | 15.5 |
| Products administered | |
| Antiemetics and antinauseants | 59.1 |
| Immunostimulants | 43.1 |
| Intravenous solutions | 43.1 |
| Contrast agents for resonance imaging | 43.1 |
| Local anesthetics | 40.8 |
| Propellants | 36.4 |
| Non-iodinated contrast agents | 32.1 |
| Other anti-anemic preparations | 29.9 |
| Non-associated systemic corticosteroid | 27.4 |
| Anti-propellant agents | 247 |

TABLE 23

| Initial heart rate | % |
|---|---|
| Ventricular fibrillation (VF) | 8.0 |
| Ventricular tachycardia (VT) | 0.0 |
| Other | 79.6 |

TABLE 24

| Causes of death (for 102 cases) | % |
|---|---|
| Not known | 4.6 |
| Hypoxia | 4.2 |
| Ischemia | 3.6 |
| Other | 2.0 |
| Heart disease (non-ischemic) | 0.7 |
| Trauma | 0.7 |
| Dyskalemia | 0.5 |
| Subarachnoid hemorrhage | 0.3 |

### Group 7:

The seventh group is characterized by the following elements:

TABLE 25

| Size | Group 7 | Total population |
|---|---|---|
| Number of cases | 460 | 3.6% |
| Age | 52 | 68 |
| Male | 83% | 61% |
| Universal health insurance (CMU) | 17% | 4.1% |
| Shockable rhythm | 14.6% | 16.8% |
| Transported alive | 30.2% | 24% |
| Mortality rate (after hospitalization) | 93.9% | 94.6% |

TABLE 26

| | Deviation from |
|---|---|
| | average |
| Diagnosis | |
| Alcohol-related mental disorder | 33.9 |
| Human viral immunodeficiency | 18.9 |
| Severe liver disease | 16.8 |
| Care involving rehabilitation (including | 16.4 |
| alcohol withdrawal) | |
| Chronic viral hepatitis | 16.1 |
| Tobacco addiction | 15.3 |
| Difficulties related to economic or housing | 14.0 |
| conditions (including homelessness) 14.0 | |
| HIV in asymptomatic phase | 11.9 |
| Epilepsy | 11.4 |
| Drowsiness, stupor and coma | 11.4 |
| Products administered | |
| Drugs for addictive disorders | 35.9 |
| Direct-acting antivirals | 22.8 |
| Antiepileptic drugs | 9.1 |
| Antipsychotic drugs | 8 |
| Sulfonamides and trimethoprim | 5.9 |
| Anxiolytics | 5.6 |
| Other nutrients | 3.9 |
| Hypnotics and sedatives | 3.6 |
| Drugs for amoebiasis and other protozoa | 2.7 |
| Immunostimulants | 2.6 |

TABLE 27

| Initial heart rate | % |
|---|---|
| Ventricular fibrillation (VF) | 13.9 |
| Ventricular tachycardia (VT) | 0.7 |
| Other | 77.3 |

TABLE 28

| Causes of death (for 145 cases) | % |
|---|---|
| Not known | 9.8 |
| Hypoxia | 9.3 |
| Ischemia | 5.3 |
| Other | 2.0 |
| Trauma | 1.7 |
| Subarachnoid hemorrhage | 1.1 |
| Heart disease (non-ischemic) | 0.9 |
| Pulmonary embolism | 0.7 |
| Drug poisoning | 0.2 |

### Algorithm for Predicting the Occurrence of Sudden Death

Within the framework of these experiments, the Applicant developed an algorithm to predict the occurrence of sudden adult death within a 1-year time window, based on care trajectories observed over a 5-year period before the event.

To do this, the Applicant compared the performance of different supervised statistical classification techniques.

To do this, the Applicant trained and compared each of the techniques using a 10-part consolidated cross-validation.

Specifically, the Applicant iteratively divided the data into two sets: a training set and a test set with a ratio of 9 to 1 (for every 10 available data, 9 are used for training and 1 for testing. In addition, the sets are modified so that the proportion of sudden death (sometimes referred to as SCD for Sudden Cardiac Death) in a set is the same in each set.

The Applicant then calculated the Area Under Curve (AUC), the Positive Predictive Value (PPV) and the Sensitivity.

The techniques compared are 3 techniques, namely:
a first technique T1: logistic regression,
a second technique T2: decision trees, and
a third technique T3: K nearest neighbors.

Tables 29 to 32 give the performance of 4 models for a prediction of sudden death at one year.

The first model M1 corresponds to the SCD comparison with the general population, the second model M2 corresponds to the SCD comparison with acute myocardial infarction (more often referred to by the acronym AMI), the third model M3 corresponds to the SCD comparison with chronic heart failure (more often referred to as HF) and the fourth model M4 corresponds to the SCD comparison with ischemic heart disease (more often referred to as IHD).

The results obtained are as follows:

TABLE 29

| Performance | T1 | T2 | T3 | Invention |
|---|---|---|---|---|
| AUC | 0.84 | 0.85 | 0.76 | 0.87 |
| PPV (%) | 75% | 81% | 74% | 83% |
| Sensitivity (%) | 85% | 83% | 73% | 86% |

### Performance for the First Model M1

TABLE 30

| Performance | T1 | T2 | T3 | Invention |
|---|---|---|---|---|
| AUC | 0.77 | 0.72 | 0.65 | 0.81 |
| PPV (%) | 73% | 71% | 68% | 75% |
| Sensitivity (%) | 74% | 67% | 57% | 79% |

### Performance for the Second Model M2

TABLE 31

| Performance | T1 | T2 | T3 | Invention |
|---|---|---|---|---|
| AUC | 0.82 | 0.83 | 0.75 | 0.86 |
| PPV (%) | 74% | 79% | 72% | 82% |
| Sensitivity (%) | 81% | 81% | 71% | 86% |

### Performance for the Third Model M3

TABLE 32

| Performance | T1 | T2 | T3 | Invention |
|---|---|---|---|---|
| AUC | 0.81 | 0.82 | 0.73 | 0.85 |
| PPV (%) | 72% | 78% | 71% | 80% |
| Sensitivity (%) | 80% | 78% | 70% | 82% |

### Performance for the Fourth Model M4

None of these techniques were satisfactory, so the Applicant turned to a neural network technique.

The training was done on each of the above-mentioned sets of data with the same four previous models.

From this, the Applicant selected a recurrent neural network with gates.

This allows to obtain for each of the above mentioned models much better area under the curve, positive prediction PPV and sensitivity performances.

To further improve these results, the Applicant used the 7 groups determined by the above classification technique and used data augmentation techniques to artificially increase the number of sudden death cases in each group during the training phases of the neural network. This compensates for the imbalance in each group (1 sudden death case for 12 control individuals).

For data augmentation, the Applicant used a generative adversarial network. Such a network is more often referred to by the acronym GAN, which refers to the corresponding English name of “Generative Adversarial Networks”. The Applicant has indeed observed that a GAN network is well adapted for the generation of medical data.

An algorithm is thus obtained for predicting the risk of occurrence of sudden death in a patient for each group. The output of the algorithm developed by the Applicant is then a quarterly score (there are thus 4 risk scores for a time window of 1 year) and adapted according to the population (one of the 7 groups) to which the individual belongs.

The Applicant also used an algorithm to interpret the results. In this case, the algorithm selected by the applicant is the SAE, an abbreviation that refers to the name “Shapley Additive Explanation” literally meaning additive explanation of Shapley. This provides the most important risk factors that explain these predictions for each individual.

1. A method for predicting the risk of the occurrence of a sudden death in a patient, the method being computer implemented and comprising the steps of:
receiving data relating to the care pathway of a patient,
determining, from the data relating to the care pathway of the patient, whether the patient belongs to one of a set of predefined groups, each group being associated with a set of predefined data, to obtain a determined group and a set of predefined determined data,
searching, for each predefined determined data, of the value of the predefined data of the patient, to obtain a set of values specific to the patient, and
applying a neural network to the patient-specific values to obtain a risk of the occurrence of sudden death for the patient, the neural network being specific to the determined group.

2. The prediction method according to claim 1, wherein each neural network is a recurrent gated neural network.

3. The prediction method according to claim 1, wherein the received data 20 is data relating to the care pathway of the patient during the previous five years.

4. The prediction method according to claim 1, wherein each predefined data is the presence of a disorder or the intake of a drug.

5. The prediction method according to claim 1, wherein the number of predefined data of a group is between 10 and 30.

6. The prediction method according to claim 5, wherein:
a group is associated with predefined data relating to a breathing disorder or to the intake of a breathing disorder limiting product,
a group is associated with predefined data relating to a neurological disorder or the intake of a neurological disorder limiting product,
a group is associated with predefined data related to a cancer disorder or to the intake of a cancer disorder limiting product,
a group is associated with predefined data related to an addiction disorder or to the intake of an addiction disorder limiting product,
a group is associated with predefined data related to an aging disorder or to the intake of an aging disorder limiting product, and
a group is associated with predefined data related to a cardiac disorder or the intake of a cardiac disorder limiting product.

7. The prediction method according to claim 1, wherein the groups are obtained by applying a k-means partitioning technique on a set of data comprising data related to the care pathway of a set of patients and each predefined data of a group is obtained by applying a language analysis tool on a set of data comprising data related to the care pathway of the set of patients.

8. The prediction method according to claim 7, wherein the set of data includes 15 artificial data generated by applying a function to data relating to the care pathway of a set of patients.

9. The prediction method according to claim 8, wherein the function is an adversarial neural network. 20

10. A computer program product including program instructions forming a computer program stored on a readable information medium, the computer program being loadable onto a data processing unit and implementing an evaluation method according to claim 1 when the computer program is implemented on the data processing unit.

11. A readable information medium including program instructions forming a computer program, the computer program being loadable onto a data processing unit and implementing an evaluation method according to claim 1 when the computer program is implemented on the data processing unit.

12. The prediction method according to claim 1, wherein the number of predefined data of a group is between 15 and 25.

13. The prediction method according to claim 1, wherein the number of 25 predefined data of a group is equal to 20.
